(19) United States
(12) Patent Application Publication
(10) Pub. No.: US 2024/0296318 A1
(43) Pub. Date: Sep. 5, 2024

(54) NEUROMONITORING SYSTEMS

(71) Applicants: Thomas James OXLEY, New York, NY (US); Jason WRIGHT, Long Island City, NY (US); Cesar ECHAVARRIA, Brooklyn, NY (US)

(72) Inventors: Thomas James OXLEY, New York, NY (US); Jason WRIGHT, Long Island City, NY (US); Cesar ECHAVARRIA, Brooklyn, NY (US)

(21) Appl. No.: 18/657,409

(22) Filed: May 7, 2024

Related U.S. Application Data
(63) Continuation of application No. PCT/US2023/069512, filed on Jun. 30, 2023.
(60) Provisional application No. 63/367,592, filed on Jul. 1, 2022.
(60) Provisional application No. 63/502,831, filed on May 17, 2023.
(60) Provisional application No. 63/506,152, filed on Jun. 5, 2023.

(51) Int. Cl. G06N 3/06; G06F 3/01

(52) U.S. Cl. CPC G06N 3/061; G06F 3/015

(57) ABSTRACT

Brain-computer interface (“BCI”) systems that provide paralyzed individuals with more meaningful autonomy and independence. Including BCI systems used by an individual that requires less assistance from, or even in the absence of, a care giver, BCI systems that provide an objective and functional measurement of the effectiveness of a motor neuroprostheses in restoring motor outputs.

### CROSS-REFERENCE TO RELATED APPLICATIONS

This application is a continuation of PCT Application No. PCT/US2023/069512 filed Jun. 30, 2023, which claims priority to U.S. provisional application No. 63/367,592, filed Jul. 1, 2022, U.S. Provisional application No. 63/502,831, filed May 17, 2023, and U.S. Provisional application No. 63/506,152, filed Jun. 5, 2023, the entirety of each application is incorporated by reference.

### BACKGROUND

It is presently understood that in most cases, a human brain functions as a well-coordinated network made up of collections of various brain regions, each an individual network of brain tissue and cells that is responsible for a specific purpose. Presently, statistical analysis of the functional magnetic resonance imaging (“fMRI”) allows neuroscientists to map the regions of the brain responsible for specific tasks. Furthermore, it is understood that many cognitive tasks are performed by the networking of several discrete brain regions that are “functionally connected”. Therefore, the brain can be considered a distributed neural network that coordinates a series of sub-networks associated with various regions of the brain, where each subnetwork is associated with a specific purpose.

Presently, conventional approaches exist that attempt to access these regions of the brain. Such approaches generally known include deep brain stimulation (“DBS”), which involves implanting electrodes within certain areas of a brain where the electrodes produce electrical impulses in an attempt to stimulate or regulate brain activity for therapeutic or other purposes, as well as electrocorticography (“ECoG”), which enables neuromonitoring of brain regions for a diagnostic purpose.

DBS involves creating small holes in the skull to implant the electrodes and surgery to implant a controller or pacemaker-like that is electrically coupled to the electrodes to control the stimulation. Typically, this device is positioned under the skin in the chest. The amount of stimulation in deep brain stimulation can be controlled by the controller or pacemaker-like device where a wire/lead connects the controller device to electrodes positioned in the brain.

DBS can be used to treat a number of neurological conditions, such as tremors, Parkinson's disease, dystonia, epilepsy, Tourette syndrome, chronic pain, and obsessive-compulsive disorder. In addition, Deep brain stimulation has the potential for the treatment of major depression, stroke recovery, addiction, and dementia. Moreover,

ECoG can provide a means of recording high-fidelity brain activity, for instance, during operations (intraoperative neuromonitoring), where real-time brain activity recordings may enable the treating surgeon to make immediate decisions that improve the safety of the treatment. Longer-term recordings are utilized for seizure detection in epilepsy and to assist in mapping to improve the safety of tumor resection by limiting removal of healthy brain tissue. However, ECoG requires placement of electrode arrays directly onto the surface of the brain following exposure of the brain via craniotomy, for example, with subdural or epidural arrays. Therefore, their use is extremely limited in application.

FIG. 1 illustrates a conventional approach of accessing regions of the brain with a brain stimulation device 20 containing electrodes 22 that are implanted within a brain 12 of an individual 10. As shown, the implantation requires surgical penetration of the cranium 14 by the device 20 such that the device 20 is directed towards an area of interest 30. In addition, a lead 16 couples the device 20 to a controller/transceiver/generator 18.

There are a number of risks associated with the general surgery required to surgically implant the device 20 in conventional DBS procedures. Furthermore, there are risks in the process of the DBS procedure itself, given that conventional procedures require an approximation or non-invasive attempt to locate the region of interest 30. Then, the physician must attempt to physically position the electrodes 22 of the device 20 in or near the area of interest 30 such that the desired effect can be achieved. In certain cases, the positioning of the electrodes 20 can be a trial-and-error approach requiring multiple surgical attempts and multiple surgical insertion sites. Regardless of the number of attempts, the act of inserting the device 20 to position the electrodes 22 in the area of interest 30 creates collateral damage to brain tissue located in the path between the area of interest and the insertion point in the cranium.

Currently, the surgical risks involved in such procedures can include bleeding in the brain, stroke, infection, collateral damage to brain tissue, collateral damage to vascular structures in the brain, temporary pain, and inflammation at the surgical site. Apart from the surgical risks, DBS involves risks in side effects of DBS if the electrodes stimulate or affect areas outside of the area of interest 30. Such risks can include breathing problems, nausea, heart problems, seizures, headache, confusion, etc. Yet an additional risk can be introduced upon attempting to remove a DBS device after a period of time, given that tissue can heal around the device and implantation site.

However, the conventional approaches intended to access the many subnetworks of the brain are deficient such that the conventional approaches are unable to maximize the benefit of accessing and directly communicating/stimulating these subnetworks.

Conventional invasive approaches that involve direct brain penetration result in ongoing scar formation due to gliosis. Due to the nature of the level of invasiveness of craniotomy and the progressive nature of scar build-up due to gliosis it is not feasible to remove and replace conventional DBS electrodes in the brain.

In addition to the issues discussed above, the development of brain-computer-interface (“BCI”) technologies presently focuses both on safety and enabling a paralyzed person to use a BCI system to control electronic devices, including prosthetic arms and computers, and so to complete a variety of daily tasks. The need to restore continuous and independent motor outputs that allow for computer control in people living with full or partial paralysis. BCI systems hold promise for restoring lost neurologic function, including motor neuroprostheses (MNPs), to restore motor capability to the individual. An implantable MNP can directly infer motor intent by detecting local brain signals and transmitting the motor control signal out of the brain to generate a motor output and subsequently control computer actions or to control other electronic devices. In one variation, this physiological function can be performed by the motor neurons in the individual.

However, while traditional BCI systems provide a paralyzed individual with some level of autonomy through control of the device, the BCI systems that exist today afford only very limited overall autonomy to individuals with paralysis. For example, a paralyzed individual using a BCI system typically requires assistance with: setting up or turning on the BCI system (including charging/recharging); calibrating the system for an individual's use, including learning how an individual must think so to enable useful electronic commands to be generated; and setting up the individual (adjusting the screens, antennas and/or posture, etc. of the individual to allow them to use the system—as opposed to setting up the system itself).

There is a need for a BCI system that provides paralyzed individuals with more meaningful autonomy and independence. Such meaningful autonomy and independence can be provided by a BCI system used by an individual who requires less assistance from, or even in the absence of, a care giver. There also remains a need for objective and functional measures of the effectiveness of MNPs in restoring motor outputs. One such way is to rely on the concept of digitized motor outputs (DMOs), which is a motor output decoded directly from a neural recording when the individual attempts a limb movement, and the BCIU transfers this neural intent into a command that controls an electronic device. DMOs can be categorized as discrete and continuous representations of motor control, and with varying degrees-of-freedom.

The imperative for a patient's at least intermittent independence can also benefit the patient where a caregiver does not understand the patient's needs or to communicate with a third party regarding the individual's dissatisfaction with or fear of a caregiver.

### SUMMARY

The ability to access the functionally distributed network of the brain allows for directly accessing, monitoring, and/or communicating with specific regions of the brain can allow for technological improvements in a number of areas, including but not limited to healthcare, quality of life, improvements in the use of technology by an individual and improvements in the ability to communicate within a networked group of individuals. For example, direct access to this neural distributed network allows for improvement of conventional healthcare procedures for an individual and/or improvement of machine control by the individual. In an additional variation, the ability to directly access, monitor, and/or communicate with an individual's neural distributed network allows for improved communication with that individual and/or between individuals whose respective neural distributed networks are configured to directly network.

The present disclosure includes one or more methods of adjusting a neural interface by monitoring a neural activity of an individual where the neural interface comprises a neural monitoring device operatively connected to a control unit, where control unit is configured to produce an output control signal for interacting with an external electronic device, the method comprising. For example, such a method can include providing a signal from the neural monitoring device implanted within the individual to the control unit, where the signal represents the neural activity of the individual and where detection of a predetermined neural activity causes the control unit to produce the output control signal; determining an activity level of the individual by monitoring the neural activity of the individual; and wherein the control unit is configured to adjust the neural interface from a first operational configuration to a second operational configuration upon determining that the activity level meets a first predetermined condition, wherein a power consumption of the neural interface in the first operational configuration differs from the power consumption of the neural interface in the second operational configuration.

Variations of the method include the first predetermined condition that comprises a sleep status of the individual and, where determining the activity level comprises determining the sleep status of the individual by assessing the neural activity for neural sleep indicators. The first predetermined condition can comprise a failure to generate the output control signal within a pre-defined period of time.

In additional variations, the control unit can be further configured to adjust the neural interface from the first operational configuration to the second operational configuration when the output control signal is associated with an idle command instruction.

The neural interface can be configured to provide perceivable feedback to the individual to indicate whether in the first operational configuration or the second operational configuration. The neural interface can be configured to allow the individual to cause the neural interface to remain in the first operational configuration. Variations of the control unit can be further configured to transmit an idle signal to the external electronic device on or before adjusting to the second operational configuration.

Another variation of the method includes determining the activity level of the individual by monitoring the neural activity of the individual while the neural interface is in the second operational configuration and where the control unit is further configured to adjust the neural interface from the second operational configuration to the first operational configuration upon determining the activity level meets a second predetermined condition.

The control unit can be further configured to adjust the neural interface from the second operational configuration to the first operational configuration when the output control signal is associated with an active command instruction. The control unit can be further configured to adjust the neural interface from the second operational configuration to the first operational configuration when the individual generates at least one output control signal.

Another variation of a method described herein includes a method of altering a frequency of communication in a neural interface by monitoring a neural activity of an individual where the neural interface comprises a neural monitoring device operatively configured to communicate with a control unit at a communication rate, and where control unit is configured to produce an output control signal for interacting with an external electronic device.

One example of such a method includes providing a signal from the neural monitoring device implanted within the individual to the control unit, where the signal represents the neural activity of the individual and where detection of a predetermined neural activity causes the control unit to produce the output control signal; determining an activity level of the individual by monitoring the neural activity of the individual; and wherein the control unit is configured to alter the neural interface from a first operational configuration to a second operational configuration upon determining that the activity level meets a first predetermined condition, wherein the communication rate in the first operational configuration differs from the communication rate in the second operational configuration.

Another example of a method under the present disclosure includes methods of increasing an autonomy of a paralyzed individual to operate an external electronic device by providing a brain-computer interface configured to monitor a neural activity of the paralyzed individual where the brain-computer interface comprises a neural monitoring device operatively connected to a control unit, where the control unit is configured to produce an output signal for interacting with the external electronic device; where the brain-computer interface is configured to enter an idle mode wherein the brain-computer interface draws less power than in an active mode; and receiving an activation signal from the paralyzed individual to switch the brain-computer interface to an active mode from the idle mode and without assistance from a caregiver.

In such methods coupling the control unit and the external electronic device can include reducing a calibration time for the brain-computer interface when entering the active mode. The control unit and the external electronic device can be coupled wirelessly.

Variations of the method include a brain-computer interface that is configured for coupling to a re-charging supply by the paralyzed individual, or is configured to transmit operational data from the brain-computer interface to a remote electronic dashboard, where the remote electronic dashboard permits an individual to monitor activity of the brain-computer interface. The brain-computer interface can be configured to transmit operational data from the brain-computer interface to the remote electronic dashboard wirelessly. The brain-computer interface can be configured to have a latency of five seconds or less (however any larger duration is within the scope of this disclosure).

Another method of increasing an autonomy of a paralyzed individual to operate an external electronic device can include providing a brain-computer interface configured to monitor neural activity of the paralyzed individual where the brain-computer interface comprises a neural monitoring device operatively connected to a control unit, where control unit is configured to produce an output control signal for interacting with the external electronic device in response to neural activity of the paralyzed individual that is associated with a cue signal generated by the brain-computer interface; and calibrating the brain-computer interface within a minimum time period, where calibrating the brain-computer interface permits the paralyzed individual to activate the output control signal after a period of non-use of the brain-computer interface by the paralyzed individual.

Yet another variation of a method of increasing an autonomy of a paralyzed individual to operate an external electronic device can include providing a brain-computer interface configured to monitor neural activity of the paralyzed individual where the brain-computer interface comprises a neural monitoring device operatively connected to a control unit, where control unit is configured to produce an output control signal for interacting with the external electronic device when the paralyzed individual generates neural activity in response to a cue produced by brain-computer interface, where the brain-computer interface comprises a high accuracy interface ratio, where the high accuracy interface ratio is a measurement of intentional neural activity associated with the cue to neural activity not associated with the cue. The high accuracy interface ratio comprises 95%.

The present disclosure also includes brain-computer interfaces for increasing an autonomy of a paralyzed individual when operating an external electronic device. Examples of such brain-computer interfaces can include a neural monitoring device configured to detect neural activity from the paralyzed individual; a control unit operatively connected to the neural monitoring device, where control unit is configured to produce a cue on a display where the cue is associated with one or more commands, wherein when the paralyzed individual generates neural activity associated with an intent to select the cue, the control unit generates an output control signal for interacting with the external electronic device; and wherein the control unit is configured to produce the cue and the output control signal within a minimum time.

Another example of a brain-computer interface system for increasing an autonomy of an individual to interact with an electronic device, where the individual fully or partially paralyzed, the brain-computer interface system includes a neural monitoring device coupled to the individual and configured to detect a neural activity from the individual; a control unit operatively coupled to the neural monitoring device and removably coupled to the individual, where the control unit is configured to interact with the electronic device when the individual generates the neural activity; and wherein the neural monitoring device is further configured to electronically communicate with an electronic network, such that when the control unit is decoupled from the individual, the individual remains able to communicate with the electronic network using the neural activity. Examples of neural monitoring devices include an implant configured to be positioned adjacent to or in neural tissue and an internal unit in electrical communication with the implant. Alternatively, the device can be an external device, a device that is percutaneously positioned, vascularly positioned, or a combination of any of the preceding.

In one variation, the brain-computer interface system includes a neural monitoring device that is configured to electronically communicate with the electronic network independently of the control unit. Alternatively, or in combination, the neural monitoring device is configured to electronically communicate with the electronic network using the control unit.

Another variation of a brain-computer interface system for increasing an autonomy of an individual to interact with an electronic device, where the individual fully or partially paralyzed, includes a neural monitoring device coupled to the individual and configured to detect a neural activity from the individual; a control unit operatively coupled to the neural monitoring device and removably coupled to the individual, where the control unit is configured to interact with the electronic device when the individual generates the neural activity; and wherein the control unit is further configured to electronically communicate with an electronic network, such that when the electronic device is decoupled from the individual, the individual remains able to communicate with the electronic network using the neural activity.

The present disclosure also includes methods of increasing an autonomy of an individual using a brain-computer interface, where the individual fully or partially paralyzed that include positioning a neural monitoring device in or on the individual, the neural monitoring device configured to detect a neural activity from the individual; decoupling a control unit from the individual, where the control unit is configured to operatively couple to the neural monitoring device and is configured to interact with one or more electronic devices when the individual generates the neural activity; and coupling the neural monitoring device to an electronic network such that the individual maintains an ability to communicate with the electronic network using the neural activity.

The method can further include coupling the neural monitoring device to the electronic network by coupling the neural monitoring device to the electronic network using the control unit when the control unit is disengaged from the one or more electronic devices.

In another variation, the method can include coupling the neural monitoring device to the electronic network by coupling the neural monitoring device to the electronic network without using the control unit. Another variation includes coupling the neural monitoring device to the electronic network but decoupling a control unit from the individual.

In any of the methods above, the neural monitoring device can comprise an implant that is configured to be positioned adjacent to or in neural tissue and an internal unit in electrical communication with the implant.

### BRIEF DESCRIPTION OF THE DRAWINGS

FIG. 1 illustrates a conventional approach to accessing regions of the brain with a brain stimulation device containing electrodes that are implanted within a brain of an individual.

FIG. 2A shows an illustration of a cerebral cortex of a brain having a network of vasculature that supplies various regions of the cerebral cortex of the brain.

FIG. 2B is an illustration of the brain with the vasculature omitted to various cytoarchitecture regions of the cerebral cortex.

FIG. 3 shows a variation of an endovascular electrode array as part of a microwire monitoring/stimulation probe.

FIG. 4A illustrates a first variation of a system that directly accesses and monitors a specific region or subnetwork of a brain via a vascular approach.

FIG. 4B illustrates a variation of a system that directly accesses and monitors discrete regions or subnetworks of a brain via a vascular approach.

FIG. 5 illustrates a variation of a system comprising a plurality of microwire monitoring probes coupled to one or more monitoring devices.

FIG. 6 illustrates an application of the systems described herein using a distributed neural network of a brain for improved technology control, motor control, emotional monitoring, decision-making monitoring, sensory feed, auditory feed, visual feed, and communications to and from an individual.

FIG. 7 shows an example of a system using a distributed neural network of an individual for improved communication of data to and from the individual for improved interaction with any type of external device or machine.

FIG. 8 illustrates another variation of using a distributed neural network for improved communication of data to and from an individual for monitoring of the individual.

FIG. 9 illustrates a variation of using distributed neural networks to create a brain-to-brain network between at least two individuals.

FIG. 10 illustrates a brain-computer interface for demonstrating providing increased autonomy to an individual suffering from full or partial paralysis.

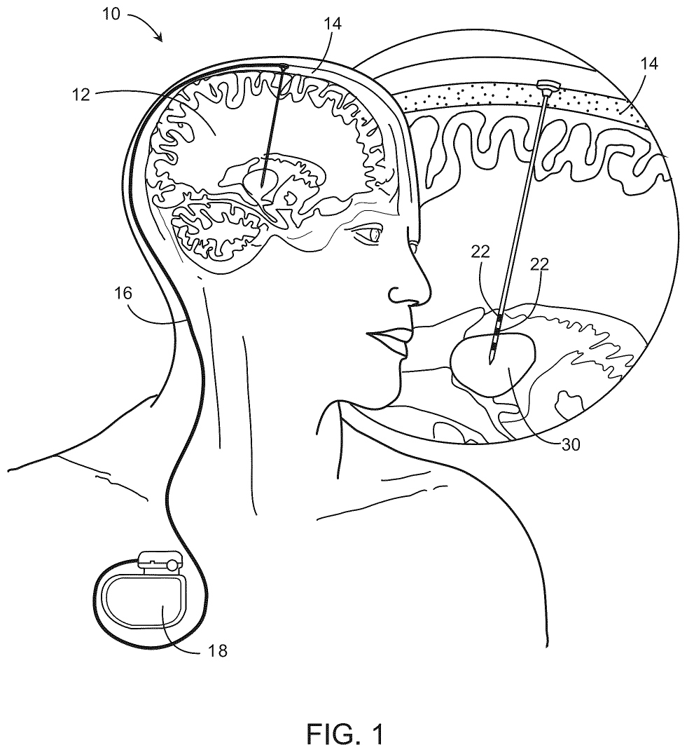

### DETAILED DESCRIPTION

The present methods and devices relate to electrodes that are configured for directly accessing, monitoring, and/or communicating with specific regions or subnetworks of the brain via a vascular approach for the purpose of using the direct access to send data to and out of the various subnetworks of a brain and associated nerves of an individual. As discussed below, the use of such data that is directly communicated to/from these neural subnetworks can improve any number of areas, including but not limited to medical applications, control of machines and electronic devices, real-time feedback on a goal-oriented activity, as well as communication and consumer goods.

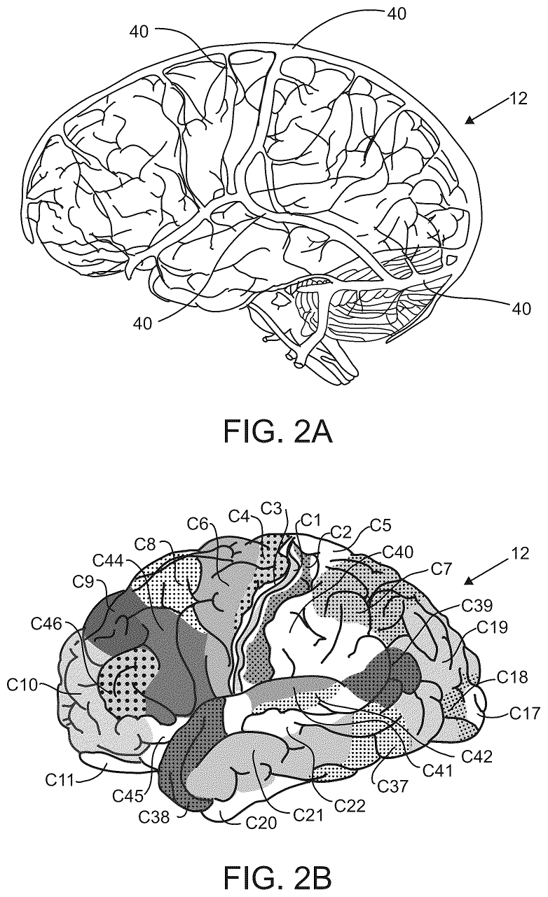

FIG. 2A shows an illustration of a cerebral cortex of a brain 12 having a network of vasculature 40 that supplies various regions of the cerebral cortex of the brain 12. The methods and devices described herein use the vasculature 40 to position one or more electrodes adjacent to a particular region of the brain. Variations of the methods can include using veins and/or arteries for positioning of the devices. In certain variations, the electrodes are positioned in veins to reduce inadvertently reducing or stopping blood flow to brain tissue. Moreover, as discussed below, the devices can be positioned entirely within a vessel. However, variations can include the use of devices or structures that penetrate the wall of a vessel.

FIG. 2B is an illustration of the brain 12 with the vasculature omitted to various cytoarchitecture regions (C1 to C46) of the cerebral cortex. These regions correspond to Brodmann areas that are based on an organization of neurons that were observed in the cerebral cortex that corresponds to various cortical functions of the cerebral cortex. C1, C2, and C3 represent primary somatosensory cortex in the postcentral gyrus; C4 is the primary motor cortex; C5 is the superior parietal lobule; C6 is the premotor cortex and supplementary motor cortex; C7 is the visuo motor cortex; C8—includes frontal eye fields; C9—dorsolateral prefrontal cortex; C10—anterior prefrontal cortex (most rostral part of superior and middle frontal gyri); C11—orbitofrontal area (orbital and rectus gyri, plus part of the rostral part of the superior frontal gyrus); C17—primary visual cortex (V1); C18—secondary visual cortex (V2); C19—associative visual cortex (V3, V4, V5); C20—inferior temporal gyrus; C21—middle temporal gyrus; C22—part of the superior temporal gyrus, included in Wernicke's area; C37—fusiform gyrus; C38—temporopolar area (most rostral part of the superior and middle temporal gyri); C39—angular gyrus, considered by some to be part of Wernicke's area; C40—supramarginal gyrus considered by some to be part of Wernicke's area; C41 and C42—auditory cortex; C44 and C45—Broca's area, includes the opercular part and triangular part of the inferior frontal gyrus; AND C46—dorsolateral prefrontal cortex.

The devices, methods, and systems described herein can benefit or be combined with endovascular carriers and electrode arrays and systems/methods of using neural signals disclosed in U.S. patent Nos.: U.S. Pat. No. 10,575,783 issued on Mar. 3, 2020; U.S. Pat. No. 10,485,968, issued on Nov. 26, 2019; U.S. Pat. No. 10,729,530 issued on Aug. 4, 2020; and U.S. Pat. No. 10,512,555 issued on Dec. 24, 2019. U.S. Publication Nos.: US20190358445, published on Nov. 28, 2019; US20180303595, published on Oct. 25, 2018; US20200352697, published on Nov. 12, 2020; US20190038438 published on Feb. 7, 2019; US20200078195 published on Mar. 12, 2020; US20190336748 published on Nov. 7, 2019; US20200016396 published on Jan. 16, 2020; and US20200363869 published on Nov. 19, 2020. U.S. application Ser. No. 17/093,196 filed on Nov. 9, 2020. PCT Application Nos.: PCT/US2020/060780, PCT/US2020/059509, both filed on Nov. 6, 2020. U.S. Provisional application Nos. 63/003,480 filed on Apr. 1, 2020; 63/057,379 filed on Jul. 28, 2020; and 63/062,633 filed on Aug. 7, 2020. The contents of each of which are incorporated herein by reference in their entireties.

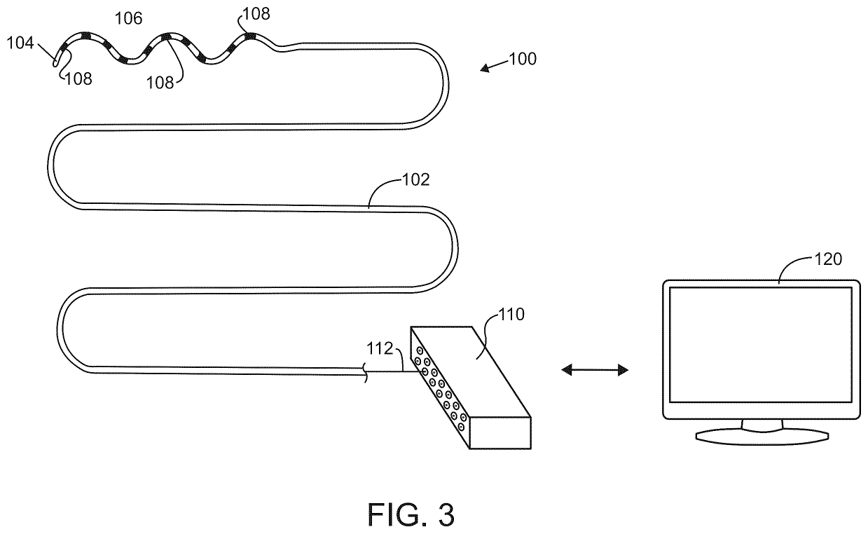

FIG. 3 shows an additional variation of an endovascular electrode array as part of a microwire monitoring/stimulation probe 100. As shown, the probe 100 includes one or more distal electrodes 108 located on a helical or sinusoidal 106 portion of a microwire 102. As discussed below, the non-linear distal portion 106 comprises an atraumatic tip 104 that allows for temporarily securing the electrodes 108 and distal portion 106 within a vessel within the brain without causing trauma to the vessel or brain. The non-linear shape 106 can function to provide apposition of the wire against the vessel wall as well as position the electrodes 108 in contact with a vessel wall. The non-linear distal portion 106 can comprise a Nitinol material or core that allows the device to assume the non-linear shape when unrestrained or activated with current. Examples of microwires can be found in U.S. Pat. Nos. 6,260,458; 6,428,489; 6,431,039; 6,440,088; 6,553,880; 6,579,246; and 6,766,720, the entirety of each of which is incorporated by reference.

In additional variations, an entirety of the microwire 102 can comprise a shape memory alloy. In most variations, the device 100 is configured to be removable from the vessel, for example, by pulling on the proximal end of the microwire 102. Additional variations of the device 100 include a non-linear shape at the electrode region 106 that can range from a helical shape to a simple bend or any shape that allows for anchoring in the delicate vessels of the brain. Alternatively, the series of electrodes 108 can be positioned on any structure that provides anchoring but does not restrict blood flow within the vessel.

The microwire 102 is typically sized in length and diameter such that it can be advanced into remote vasculature within the brain. For example, the diameter of the microwire 102 can range from 0.010 to 0.018 inches. However, the size of the microwire should be chosen to allow advancement of the electrode portion into remote areas of the brain. Alternatively, a proximal portion of the microwire 102 can have a larger diameter than the medial and distal regions to allow for increased pushability of the wire 102. The proximal end 112 of the microwire 102 is coupled to a connector base 110 that communicates using either a wireless or wired connection with monitoring software or other electronic/computing device 120.

In addition to being non-traumatic, variations of the monitoring probes 100 described herein are configured to be removable when used over a short time period. Alternatively, variations of monitoring probes can remain implanted over the span of months and/or years. In any case, the device 100 can have anti-thrombotic coatings (e.g., heparin) to inhibit clotting of blood.

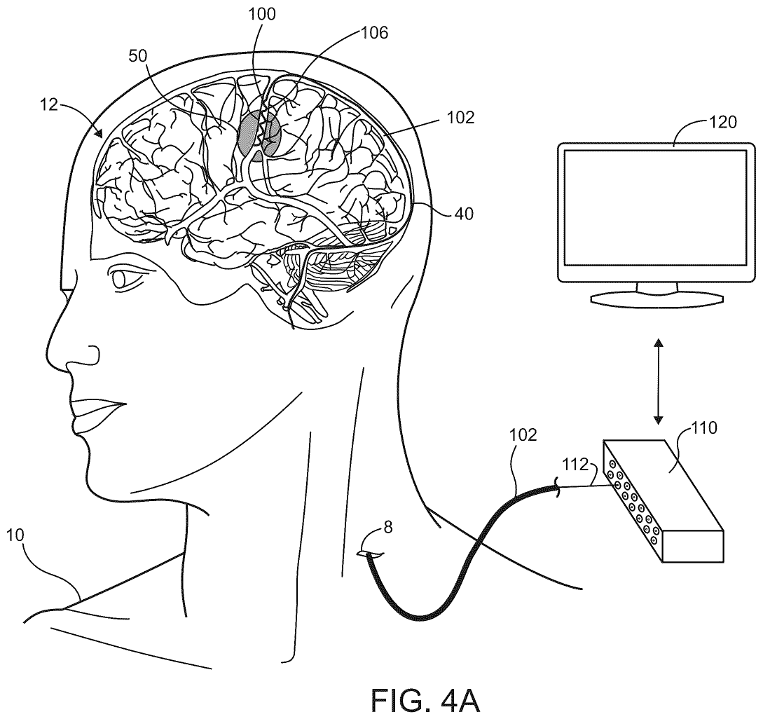

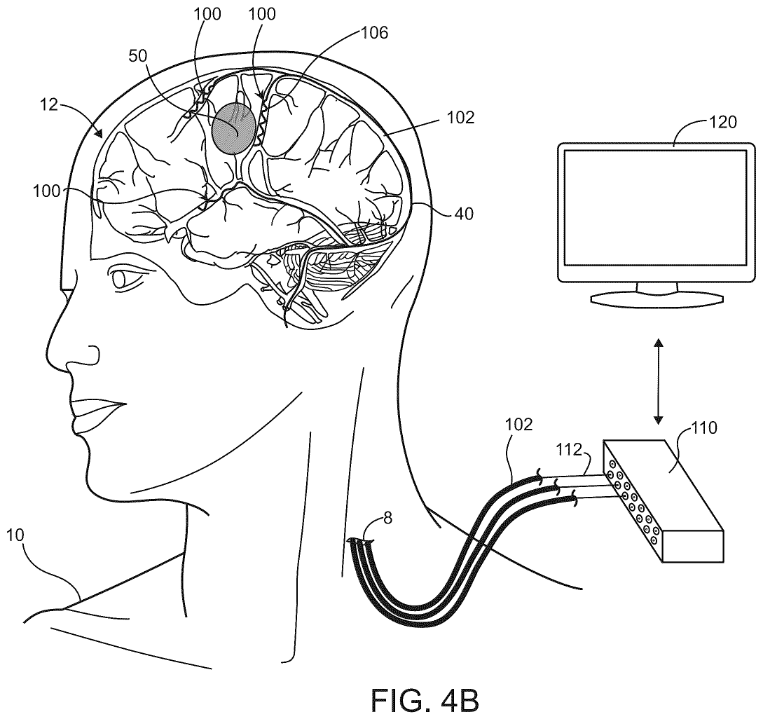

FIG. 4A illustrates a first variation of a system that directly accesses and monitors a specific region or subnetwork of a brain 12 via a vascular approach. In this variation, a microwire monitoring probe device 100 is advanced through the vasculature into a vessel 40 within the brain 12. The illustrated variation is shown to be implanted for an operative procedure to provide brain monitoring during the procedure. In this variation, the device 100 can be removed after the procedure or can remain implanted during a post-procedure monitoring period. Therefore, a proximal portion of a microwire 102 extends through one or more incisions 8 within the individual 10 for coupling to a controller 110 or another connector base. The system shown in FIG. 4A includes a single device 100 for purposes of illustration. In practice any number of microwire monitoring probe devices can be used. Moreover, the device 100 can be positioned using a microcatheter (not shown) that restrains the electrode portion 106 until deployed. Alternatively, a caregiver can directly advance the device 100 in a linear configuration. Once positioned within a desired region, the caregiver can apply a current to the device to transform the electrode portion 106 from a linear configuration into the non-linear configuration so that the device remains anchored where desired.

As shown, a distal portion of the device 106 is configured to detect neural activity as well as remain temporarily anchored within a vessel. The device 100 is deployed within a vessel and adjacent to a region of interest 50. In this example, the region of interest 50 represents an area of brain tissue that is intended to be removed or inactivated. Such procedures may involve tumor removal, removal of brain tissue to reduce epileptic seizures, treatment of arteriovenous malformations in the brain, etc. In conventional approaches, dye is used to identify the target region 50. Positioning one or more devices 100 in vessels adjacent to or surrounding the target region 50 allows for monitoring of neural signals at the site of the device deployment. The neural signals can be monitored before, during, and after injection of the dye to see the effect of the dye or the procedure.

In additional variations, as shown in FIG. 4B, the devices described herein can replace or augment a Wada test that is performed on epilepsy patients considering surgery. The Wada test, also known as the intracarotid sodium amobarbital procedure (ISAP), establishes cerebral language and memory representation of each hemisphere. In the tests, which are conducted with the patient awake, a medical practitioner introduces a barbiturate (e.g., sodium amobarbital) into one of the internal carotid arteries via a cannula or intra-arterial catheter. The medical practitioner injects the drug into one hemisphere at a time into the right or left internal carotid artery to inhibit the respective side of the brain. For example, if the drug is injected into the right carotid, the right side of the brain becomes inhibited and cannot communicate with the left side of the brain. This allows the medical practitioner to observe the effect on any language and or memory function in that hemisphere in order to evaluate the other hemisphere. The test can also involve an EEG recording to confirm that the affected side of the brain is inactive. The practitioner can then engage the patient in language and memory-related tests. The present devices can allow for positioning of devices 100 within specific cytoarchitecture regions of the brain (see FIG. 2B) to record activity while the practitioner administers various memory, language, or psychological exercise that activates the brain. Detecting neural activity during the tests allows for mapping where that task is occurring from inside the brain. Once mapped, the practitioner can determine whether treatment/removal of the area of interest 50 can be performed and the potential consequences of doing so. In addition, the devices 100 can be used to monitor the various regions of the brain during the procedure as well as after the procedure.

In another variation, such as in FIGS. 4A and 4B, the devices 100 may be placed in specific cytoarchitecture regions of the brain (see FIG. 2B) to aid in the safe intraarterial or intravenous embolization of arteriovenous malformations or malignancy. The use of neuromonitoring during endovascular embolization procedures involves the recording of evoked potentials from the brain using scalp-based EEG techniques. A somatosensory evoked potential is triggered by an electrical impulse delivered via electrical stimulation of the lower limb and recorded from the sensory cortex via EEG. To ensure the artery targeted for embolization is not supplying normal brain, an anesthetic agent such as lidocaine is injected into the artery. Any reduction in evoked potentials recorded during or immediately after the injection of lidocaine may be representative of potential brain injury that may occur, should an embolization agent be subsequently injected into said target artery. Intracranial endovascular ECoG recordings are of significantly higher sensitivity than scalp-based EEG and may represent an opportunity to improve the safety of intraoperative neuromonitoring during arterial embolization procedures.

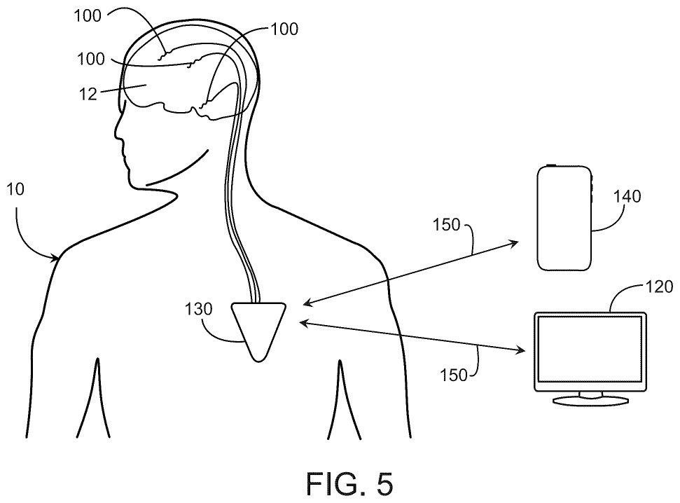

The systems described in FIGS. 4A and 4B are well suited for intraoperative and postoperative monitoring of the patient where there is little movement of the patient, such that the microwire(s) 120 can extend from one or more incisions 8 of the individual 10. FIG. 5 illustrates a variation of a system comprising a plurality of microwire monitoring probes 100 coupled to one or more monitoring devices 130. The monitoring device 130 can be fully or partially implanted within the patient 10. Alternatively, the monitoring device 130 can be positioned outside of the body but allow for coupling with the probes 100 in a sterile manner. One of the purposes of the system shown in FIG. 5 is to provide in-patient or ambulatory monitoring of an individual 10. In such cases, the probes 100 remain within specific regions of the brain 12 for days or even months.

One application of the system shown in FIG. 5 involves monitoring epilepsy patients, especially those patients that are not positively responding to medication. In such a case, any number of probes, 100, are positioned within various regions of the brain 12. The probes 100 are coupled to a monitoring device that communicates 150 either via a wired or wireless connection to any number of electronic interface devices (e.g., a personal electronic device 140 or computer system 120). The patient 10 is then taken off seizure medication for a period of time, during which the system monitors brain activity through the probes 100. The activity is then analyzed to determine the regions of the brain that are associated with the seizures, including regions that are active prior to a seizure and/or regions that are responsible for the seizure. The implanted system allows for monitoring over days or even months. Current methods of determining brain areas associated with seizures involve craniotomy surgery, which removes part of the skull or cranium to access regions of the brain. The system shown in FIG. 5 performs brain-region-seizure mapping using a vascular approach. In additional variations, the systems described herein can be used in addition to conventional procedures.

The implanted unit 130 can include amplifiers, filters, controllers, data storage, a power supply, and wireless communication equipment (e.g., RF, Bluetooth, etc.). Such equipment allows capturing of data over relatively long periods of time to provide the individual with mobility while being assessed.

In addition to brain mapping, by being implanted over a longer duration, the systems described herein can provide a warning system for patients that are subject to seizures. For example, the implants 100 can monitor various regions of the brain 12 and provide notifications via an external device (e.g., 140) or via the monitoring device 130 if the system detects that the individual is at a high risk of having a seizure. In such a case, the individual can be put on alert and avoid environments where a seizure would cause additional risk (e.g., driving, bathing, exercising, etc.). The system could also give varying levels of warning, such as low, medium, and high risk of seizure, that would allow an affected individual to have increased freedom from a sudden unexpected seizure.

In another variation, the systems described herein can also serve as a neuromonitoring diagnostic system that detects electrophysiological biomarkers in patients suffering from brain injury where the patient is otherwise unresponsive. Detection of the biomarkers can be an indicator of patient recovery. An example of such a response is discussed in Claassen, J. (2019). Detection of Brain Activation in Unresponsive Patients with Acute Brain Injury. The New England Journal of Medicine, 380(26) 2497-2505.

For example, in cases where a coma, stroke, hypoxic brain injury, or any brain injury renders the patient clinically unresponsive. The use of the systems described herein can assess the unresponsive patient for evidence of brain activation using ECoG in response to external stimuli, including auditory stimuli (e.g., spoken commands, familiar voices, etc.) and/or physical stimuli. In one variation, the purpose of the stimulus is to induce changes in brain state by interacting with the unresponsive patient. The neuromonitoring system can then provide a caregiver with a user interface/user exchange to provide various information to the caregiver regarding the condition of the patient. For example, the user interface can provide a prediction of outcome, degree of recovery, and/or measure improvement in the unresponsive patient over time. The measured response to the external stimulation can be compared to a dataset to predict recovery patterns of the patient. The dataset can be cloud-based and updated based on machine learning algorithms that provide data standardization to provide a rating of the patient's condition, such as likely to improve or unlikely to improve.

The neuromonitoring system can also be combined with provocative testing, where the patient is monitored in a resting state to determine activity and then again after an anesthesia is administered to the patient or a specific region of the patient's brain. The difference in the measured signals can be an indicator of brain function.

Use of the systems described herein as a neuromonitoring system allows for positioning of one or more endovascular electrode arrays in, for example, a motor region of the brain. However, the array(s) can be positioned in any number of regions of the brain. Implantation of the electrode array can be transitory, where the array is removed after monitoring of the patient. Alternatively, the array can be implanted over a longer term for increased monitoring of the patient. In either case, it may be desirable that the proximal end of the arrays is directly coupled to a controller/transceiver/generator that is not implanted in the patient (e.g., see FIG. 3).

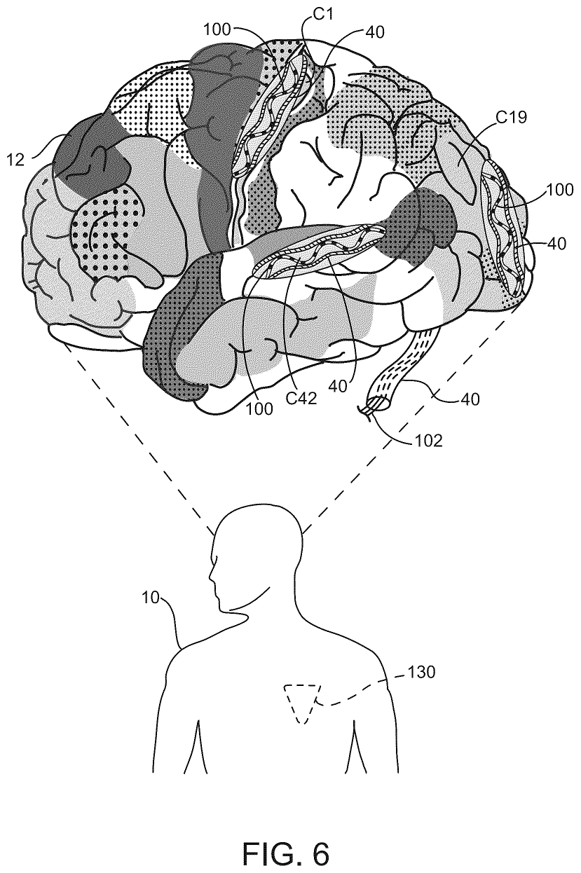

FIG. 6 illustrates another application of the systems described herein that use a distributed neural network of a brain for improved technology control, motor control, sensory feed, and communications to and from an individual. For example, FIG. 6 illustrates a magnified view of a brain 12 of an individual 10 having any number of microwire probes 100 positioned within vessels 40 associated with specific discrete cytoarchitecture regions (e.g., C1, C19, and C42) of the brain 12. Positioning the implants in discrete regions or networks of the brain allow sensory stimulation or neural signal measurement in different regions of the brain to improve communication of data to and from the individual. In the illustrated example, the cytoarchitecture regions relate to auditory, sensory, and visual regions of the brain 12. However, these regions are chosen for illustrative purposes only. Additional variations of the systems and methods disclosed herein include any number of probes 100 positioned to be associated with any number of cytoarchitecture regions of the brain.

As shown in FIG. 6, the probes 100 are coupled to a control unit 130 via microwires 102 that extend through additional vasculature 40. As discussed below, the probes 100 allow data transfer to and from the various regions of the brain 12 to provide for improved communication of information to and from the individual 10 as well as improved control of electronic devices that are networked with the probes 100 and control unit 130. The regions illustrated in FIG. 6 are useful for data being in-fed to the individual. In additional variations, the regions of the brain useful for data being out-fed from the individual can comprise the same or different regions of the brain. For example, such regions can include areas of the brain responsible for language, decision prediction, motor control, emotions, etc.

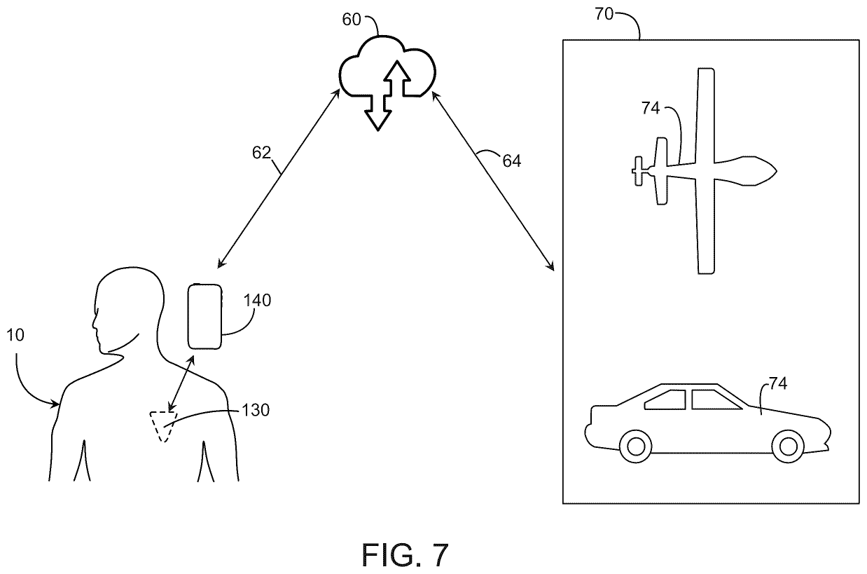

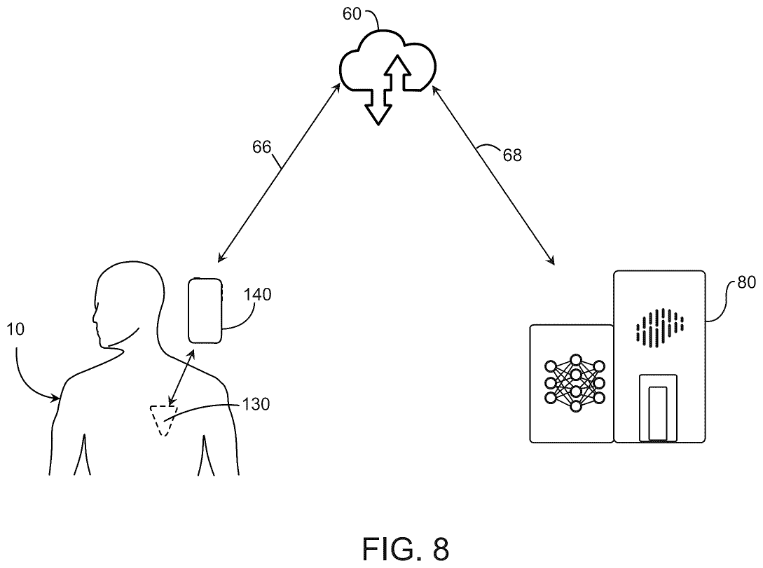

FIG. 7 shows one example of the systems described herein using a distributed neural network of an individual 10 for improved communication of data to and from the individual 10 for improved interaction with any type of external device or machine 70. FIG. 7 illustrates a plane or drone 72 and automobile 74 for purposes of explaining the improved communication or data transfer. Clearly, the disclosure can include any machine or device configured for interaction with the individual 10.

In a conventional system, an operator controls a drone using a remote-control device along with an electronic interface that includes a screen providing various data of the operational parameters of the drone (e.g., speed, altitude, fuel, direction, etc.) The operator must observe these parameters in order to respond to any changing condition of the operational parameters. Next, the operator must formulate the thought for any subsequent action, and then to enact any corrective action, the operator must carry out the physical act of providing the drone with corrective action. While the operator might perform these actions quickly, there is a time delay between a change in condition of the drone, observing the change in condition, and then carrying out the physical corrective action to control the drone. Reaction speeds for vehicle operators require thoughts to be carried from their origin in the cortex through the spinal cord, peripheral nerves and ultimately to trigger muscle activity to enact the volitional command. Device (FIG. 5) enables information transfer at a speed superior to an unmodified human body.

In a system, as shown in FIG. 7, data 64 concerning the drone's operating parameters can be transmitted to a network 62 or directly to the electronics 140 that interface with a control unit 130 of the systems described herein. (As discussed above, the use of a separate electronic unit 140 is optional for all the examples discussed herein.) As noted above (see FIG. 6), various probes can be positioned in different cytoarchitecture regions such that information 62 being transmitted to the individual can trigger stimulation of specific cytoarchitecture regions. For example, if the drone is gaining altitude the system stimulates a first region of the brain and if the drone is losing airspeed, the system can stimulate a second region of the brain. The operator will be trained to recognize the various stimulations to react accordingly. This direct transmission of data from a machine 70 to the system and individual 10 allows for a high-fidelity degree of control of the drone.

Additionally, the system can allow the individual 10 to use brain activity generated in a specific cytoarchitecture region to issue control commands to the drone. For example, if the individual 10 determines that the drone requires a course correction (e.g., move to the right), an implant positioned in a motor region of the individual will pick up brain activity of the individual who can produce a thought of a motor activity on their right side (e.g., pushing down with a right foot or activating a muscle on the right side). This neural activity is then transmitted via data 62, either through a network 60 or directly to the drone 72 such that the drone receives data 64 to automatically correct course. In both examples described herein, the system allows for direct communication between discrete regions of the brain and external machines 70 that require control. The system allows for improved control of the machines 70 as well as improved perception of the operating conditions of the machines. Although the above description discusses use of cytoarchitecture regions that control motor activity, any number of cytoarchitecture regions can be used, including but not limited to regions that control emotional broadcasting, language, decision prediction, visuospatial perception, auditory perception, and sensory perception (e.g., touch, smell, taste, etc.).

In yet a further variation, the system shown in FIG. 7 can use artificial intelligence or external data generated by a network 60 independently from the machine 70 or indirectly from the machine. For example, if an implant is positioned in a sensory region of the brain, triggering the implant can produce a perception of a smell, taste or similar sensory feed that is associated with a warning of some predetermined condition. As one example, if an individual 10 is in a hostile territory and either the drone 72 or satellites have identified areas of actual or potential risk, the implant can be triggered to generate a specific perception that is associated with the area of actual or potential risk. The perception can be triggered to increase as individual moves toward the area and decrease when moving away. Alternatively, or in addition, this additional data can be used to feed an enemy's location through a visual cortex and represented through the brain of the individual 10 on to a Geo spatial representation to produce a direct visual feed into the brain from a control station.

FIG. 8 illustrates another variation of using a distributed neural network for improved communication of data to and from an individual 10. In this variation, the implanted microwire sensor can be implanted a region of the brain corresponding to a prefrontal cortex, which is responsible for decision making. Therefore, the implanted probe can generate signals that are predictive of decision making. Such a feature can be used when the individual 10 is in a situation faced with making a difficult decision (e.g., a soldier, law enforcement, firefighter, etc.) The system can transmit data 66 68 to a monitoring site 80, which attempts to actually predict the way that the individual is making a decision and can then engage with the individual to assist, help, or even prevent the action.

In a further variation, a tactical subject on a mission with limited communications to base command, such as an astronaut, utilizes the system for superior communication (e.g., with another astronaut or Mission Control). The device (FIG. 5) enables monitoring of the real-time cognitive activity of the astronaut across the distributed cognitive domains (FIG. 2B) that aid in decision-making. For example, emotional arousal broadcasting, decision prediction, and motor function can be monitored. Mission Control or additional individuals are further able to provide information to cognitive domains of the subject that can be received in various forms of perception, including sensory, auditory, visual, and olfactory. For example, geospatial information to aid in decision-making during the mission can be provided directly into the visual cortex, and auditory feeds directly into the auditory cortex. The astronaut is then able to carry out the mission with a higher degree of precision by utilization of information flow directly into and out of cortex.

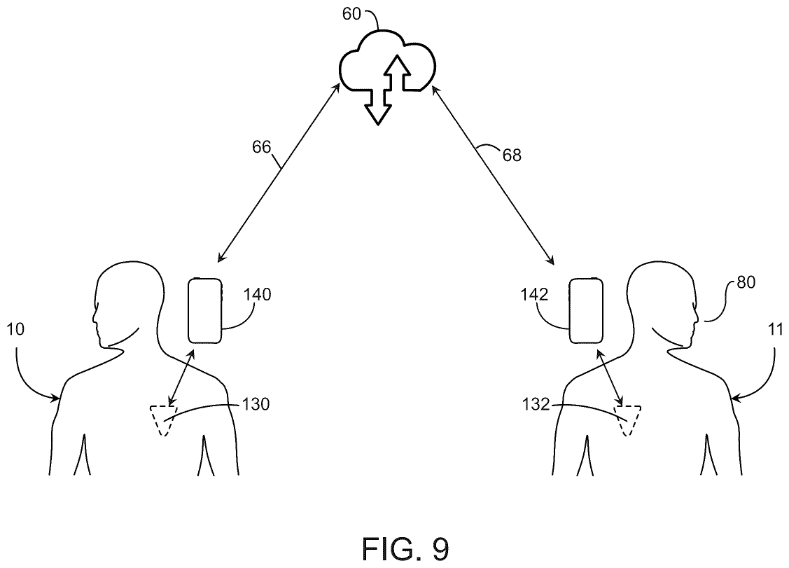

FIG. 9 illustrates another variation of using a distributed neural network by creating a brain-to-brain network between at least two individuals 10, 11 each having microwire monitoring/stimulation probes 100 respectively positioned in specific cytoarchitecture regions of their respective brain. FIG. 9 shows two individuals 10, 11 for purposes of illustration. However, the disclosure can include any number of individuals. As noted herein, the data transfer 66 and 68 between the individuals can rely on a network 60 or can occur directly through a local or private network. As also discussed, the system can include one or more electronic devices 140, 142 that communicate with a control unit 130, 132 that couples the probes, or the electronic device 140, 142 can be respectively integrated into control units 130, 132. The example shown in FIG. 9 allows linking of two individuals 10, 11 through any number of means depending on placement of the devices, in particular cytoarchitecture regions of the brain. In one example, the implants can be positioned in regions of the brain responsible for emotional responses so that each individual can be aware of an emotional component of the other. Such networking is not limited to emotions and can include connecting any region of the brain to provide sensory, motor, language, auditory, visual, taste, smell, etc. data communication directly between the individuals.

In a variation, a tactical cohort of subjects utilize networked brain function to achieve a superior level of information flow across the group. Being able to coordinate as one connected organism enables a superior group capacity to achieve a shared goal. In one example, a bright flare from an explosive may be viewed not only by a direct witness of the explosion, but by the entire group. An injury to one member of the group can be felt by the entire group. A shared consciousness across cognitive domains enables the group to perform at a higher function.

It is noted that FIGS. 7 to 9 above are illustrated as separate applications, it is with the scope of this disclosure to combine each application or portions of each application.

In addition to the applications described above, the neural interface systems described herein can provide implantable brain-computer interfaces (BCIs) for people with severe paralysis and increase their autonomy by restoring the ability to perform functions and activities of daily life with minimal intervention from a caregiver compared to current standard of care. Accordingly, the BCI systems described herein may require an “always-on” functionality. For battery-powered systems, minimizing power consumption is a priority since charging requires the assistance of a caregiver. Moreover, BCI systems may require continuously stream data in order to give the user low-latency control of target devices.

Traditionally, achieving “always-on” functionality in a battery powered system was difficult to achieve due to the high-power consumption of BCI recording hardware & decoding algorithms.

The systems described herein can reduce the concerns associated with “always-on” systems because they are already accessing the user's neural data. Therefore, variation of the systems described herein can provide different operational configurations depending on the activity of the user. For example, the operational configurations can comprise varying states of energy consumption such that one operational configuration is a low power usage configuration. The BCI can enter this low power usage state by either the user selecting this configuration using a control interface or automatically given that the system is already monitoring neural activity of the user.

For example, systems described herein can adjust a neural interface by monitoring a neural activity of an individual. The neural interface can comprise a neural monitoring device (such as an implant described herein) operatively connected to a control unit. Operatively connected can include a hardwired connection, a wireless connection, infrared, sound, and/or vibrational. The control unit can be configured to produce an output control signal for interacting with an external electronic device. Variations of the control unit include implanted control units or external telemetry units. One variation of a system and/or method includes providing a signal from the neural monitoring device implanted within the individual to the control unit, where the signal represents the neural activity of the individual and where detection of a predetermined neural activity causes the control unit to produce the output control signal; determining an activity level of the individual by monitoring the neural activity of the individual; and wherein the control unit is configured to adjust the neural interface from a first operational configuration to a second operational configuration upon determining that the activity level meets a first predetermined condition, wherein a power consumption of the neural interface in the first operational configuration differs from the power consumption of the neural interface in the second operational configuration.

In one variation, the first predetermined condition comprises a sleep status of the individual and determining the activity level comprises determining the sleep status of the individual by assessing the neural activity for neural sleep indicators. Since the BCI systems are already accessing the individual's neural signals, the systems can monitor for neural data that are not present when the user is awake. For example, neurophysiological phenomena such as K-complexes and sleep spindles occur during the earlier stages of sleep. Therefore, using neural data to detect when the user is asleep can be used to switch any device to a low-energy usage state.

Alternatively or in combination, the first predetermined condition comprises a failure to generate the output control signal within a pre-defined period of time.

The control unit can be further configured to adjust the neural interface from the first operational configuration to the second operational configuration when the output control signal is associated with an idle command instruction.

Variations of the system and/or method include a neural interface that is configured to provide perceivable feedback to the individual to indicate whether in the first operational configuration or the second operational configuration. The system and/or methods can provide a neural interface that is configured to allow the individual to cause the neural interface to remain in the first operational configuration.

In additional variations, the control unit can be further configured to transmit an idle signal to the external electronic device on or before adjusting to the second operational configuration.

A further variation of the methods and/or systems includes determining the activity level of the individual by monitoring the neural activity of the individual while the neural interface is in the second operational configuration and where the control unit is further configured to adjust the neural interface from the second operational configuration to the first operational configuration upon determining the activity level meets a second predetermined condition.

The control unit can be further configured to adjust the neural interface from the second operational configuration to the first operational configuration when the output control signal is associated with an active command instruction. In addition, the control unit can be configured to adjust the neural interface from the second operational configuration to the first operational configuration when the individual generates at least one output control signal.

Another variation of the methods and/or systems includes altering a frequency of communication in a neural interface by monitoring a neural activity of an individual. The method and/or system can include providing a signal from the neural monitoring device implanted within the individual to the control unit, where the signal represents the neural activity of the individual and where detection of a predetermined neural activity causes the control unit to produce the output control signal; determining an activity level of the individual by monitoring the neural activity of the individual; and wherein the control unit is configured to alter the neural interface from a first operational configuration to a second operational configuration upon determining that the activity level meets a first predetermined condition, wherein the communication rate in the first operational configuration differs from the communication rate in the second operational configuration.

While in the “idle” state, the BCI can perform a minimum amount of recording and computation necessary to provide a single switch output, which may be much slower than normal switch outputs generated in the “active” state. For example, in the “active” state, the BCI may be sampling all electrode channels and streaming all recorded data to the decoding algorithm. While in the “idle” state, the BCI may sample a smaller subset of electrode channels and only stream data at a lower duty cycle (e.g., 10%).

Visual and/or auditory feedback can also be provided to the user to indicate the BCI state, and this visual feedback may be synchronized with the data transmission duty cycling to make wake-up easier for the user. For example, the visual indicator may show the user that their device is currently in “idle” mode and display a countdown timer or timed radial to show the user the right moment when the wake-up switch will become available for use. In the absence of visual feedback, the user could repeatedly generate switch outputs until they observe that “active” functionality has been restored.

The wake-up switch functionality may be implemented solely in software and configurable by the user. Therefore, users can decide whether the lower operating state is desirable or not. If not, the feature can be disabled, and the BCI will always be in “active” mode.

Identification of the activity level of the user can also allow the system to control external devices that are not part of the neural interface or BCI. For example, in a smart home, the neural interface can interface with the home's control system to control various items such as turning off all lights in the house, ensuring that doors are locked, and even controlling any desired soundscapes that can enhance the quality of the user's sleep.

The BCI system can be configured so that the individual has continuous connectivity for extended periods of time. This prevents the need for a caregiver to disconnect the individual from or connect the individual to the BCI system. For example, the BCI system can comprise a charging mechanism that powers the BCI system for an increased duration of time (e.g., 6 hours). In another variation, the BCI system is configured with an “idle mode”, to reduce current drain from the battery. In the “idle mode,” the individual can activate the system or exit the “idle mode” without assistance of a care giver. In addition, allowing for ease or automatic recharging of the BCI system will increase the duration of continuous connectivity. In such a case, the BCI system allows positioning one or more components of the system into a position that allows for recharging of the component.

Another benefit of continuous connectivity and re-charging allows for minimal caregiver intervention. In such a case, a caregiver only needs to be present once per day at most to assist with charging or to perform checks on the BCI. This can be achieved by leaving the charger in place and incorporating firmware updates to the charger from a mobile app. Firmware would need to be able to turn energy on and off without a physical button push.

The aspects of the system described above all can support a patient's (at least intermittent) independence of communication.

In an additional variation, the BCI system can include one or more dashboards that provide details regarding the operating history of the BCI. Such a dashboard can comprise a monitor in electric communication with the BCI or can comprise data transmitted by the BCI to a server or network such that the operating history of the BCI is available through a portable electronic device or other website. This permits observation of the BCI operation by the individual and caregiver, but also by interested parties (e.g., relatives of the individual). This permits multiple people to monitor an individual's activity. For instance, a dashboard can show historical information about the operation of the system (e.g., when the system was in our out of an autonomous mode). While a caregiver cannot be prevented from turning the BCI off, a clinician or other family members will be able to see if part or all of the system was previously disabled on a timeline.

Variations of the BCI system described herein that allow for increased autonomy include low calibration systems, which is a measure of the ease with which the user can commence using the BCI system after a period of non-use. Calibration can be assessed quantitatively by measuring the time required for the BCI system to be calibrated to the user and made ready for use. For example, a low calibration BCI system would be one where the time between activation (e.g., switching on, leaving an idle mode) and ready for use is no more than thirty (30) seconds.

In one variation, a low calibration BCI system would include a minimal or no amount of external hardware components that need to be positioned on or to the patient and connected to the BCI system to allow the system to operate as intended. For instance, the systems described above are already implanted or coupled to the individual and can be electronically activated from an idle or off mode to start the BCI system. As noted above, a system comprising minimal components includes: a charger for the implantable receiver/transmitter unit, a SCU and a screen/device, all connected wirelessly. In contrast, a system requiring an external hardware, such as an eye-tracker device, requires physically position of a paralyzed user in front of the eye-tracker hardware.

The systems and methods described herein provide patients, including those with severe motor impairment, a return of autonomy. Accordingly, these systems and methods provide BCI's that are: continuously available; provide independent system activation; minimal calibration; an option for no wearable component or hardware that needs adjusting; and provides the individual with increased decision making.

In an additional variation, BCI systems that increase autonomy include systems having low latency, where latency is measured as the time between presenting a cue (e.g., indicating that the individual should select an action on the BCI) and the individual triggering the action (e.g., the time between presenting an option and the individual “clicking” the option). In other words, the individual is able to generate more clicks in any period of time and which support the user remaining more engaged and/engaged for longer. U.S. Provisional application No. 63/480,746, filed Jan. 20, 2023, the contents of which are incorporated by reference, discuss brain-computer interfaces, systems and methods for controlling a device based on the detection of transient oscillatory or pseudo-oscillatory bursts. Systems using detection of transient oscillatory or pseudo-oscillatory bursts can provide low latency systems.

Such autonomous BCI systems will also require high accuracy, where accuracy of the system can be measured by the rate at which a BCI system generates an action that is initiated by the individual and not initiating any action when the individual did not initiate the action.

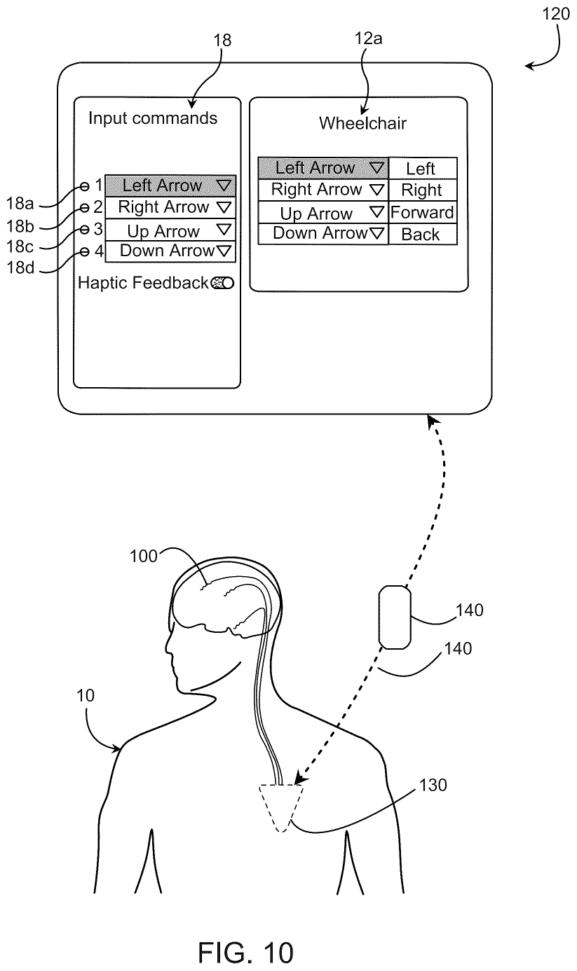

As noted above, the mechanism of action for implantable MNP in a BCI system can use detection of a motor intention in the individual and translation of that motor intention into an alternate control signal to enable the individual to perform a functionally meaningful output task. Thus, the core performance metric for an MNP and the BCI system should represent its ability to reliably translate a neural motor intention to a digital output. This may be considered a digital motor output (DMO). DMOs, carrying motor intent information, can then be mapped onto specific or generalized computer actions that can be used to control a personal computer or device. For example, as shown in FIG. 10, an implanted probe 100 and internal transmission unit 130 can translate neural motor intention of the individual 100 to an electronic device 120 that provides any number of generalized computer actions 18a-18d to directly control the device 120 or to allow the device 120 the ability to further control a secondary device 12a. In the illustrated variation, transmission 150 occurs through an external system control unit 140, however, the system can use direct transmission. The ability of an implantable MNP BCI 100/13 to produce a reliable DMO lends itself as a device agnostic approach to measurement of clinical.

The goal of evaluating the efficacy of any MNP may be obstructed by the use of performance metrics such as secondary computer actions (e.g., typing) due to variables in the operating systems and software variability of each computer action use case. For example, adding features such as predictive or generative text to an MNP operating system can lead to an inconsistent evaluation of the basic efficacy and utility of that MNP. By contrast, creating a metric structure around the DMO itself represents an implicitly valid and reliable method for evaluating the basic utility of any MNP. For example, one set of objective performance metrics that assess the extent to which any newly emerging MNP can produce DMOs and should capture the reliability of the DMO in a manner reflecting its intended application by the individual. This may be captured by assessing the accuracy of the DMO across a sufficient number of task repetitions, using a task that captures fundamental DMO performance, executed at an appropriately chosen timepoint after implant or activation. Where the accuracy is equal to the number of correct trials divided by all trials.

In an additional variation, the present disclosure includes BCI systems having subsystems that can decouple from “full system” where the full system gives added functionality/interaction for the patient, but the sub-system allows for the patient to call for help at any time. “functions in components”. For example, in FIG. 10, the full system includes the electronic device 120 (that optionally controls additional equipment or is simply a computer), an external system control unit 140 (that optionally interfaces between the individual 100 and the electronic device 120), and the implanted components e.g., 100 and 130. In practice, the system can also include additional components that enhance the ability of the individual to interact with devices but require significant time for setup/removal. For example, many BCI systems employ eye tracking equipment. The ability of the BCI system to operate as a subsystem provides the ability for the individual (especially a fully or partially paralyzed individual) with the ability to interact with a caregiver using the subcomponents, when the additional component is removed. This is especially useful when the individual decouples from the system during periods of rest or at other times when it is impractical for the individual to engage the full BCI system and components.

Accordingly, the present disclosure includes brain-computer interface systems for increasing an autonomy of an individual to interact with an electronic device, where the individual fully or partially paralysed. For example, such brain-computer interface systems can include a neural monitoring device coupled to the individual and configured to detect a neural activity from the individual. For instance, FIG. 10 shows the implant 100 and internal transmission/control unit 130 operatively coupled to a system control unit 140 that is operatively coupled to the neural monitoring device 100 and removably coupled to the individual 100.

The present disclosure also includes methods of increasing an autonomy of an individual using a brain-computer interface, where the individual fully or partially paralyzed. For example, such method can include positioning a neural monitoring device in or on the patient, the neural monitoring device configured to detect a neural activity from the individual; decoupling a control unit from the individual, where the control unit is configured to operatively couple to the neural monitoring device and is configured to interact with one or more electronic devices when the individual generates the neural activity; coupling the neural monitoring device to an electronic network such that the individual maintains an ability to communicate with the electronic network using the neural activity.

Another variation of a brain-computer interface system for increasing an autonomy of an individual to interact with an electronic device, where the individual fully or partially paralyzed, includes a neural monitoring device coupled to the individual and configured to detect a neural activity from the individual; a control unit operatively coupled to the neural monitoring device and removably coupled to the patient, where the control unit is configured to interact with the electronic device when the individual generates the neural activity; and
wherein the control unit is further configured to electronically communicate with an electronic network, such that when the electronic device is decoupled from the individual, the individual remains able to communicate with the electronic network using the neural activity.

The system control unit 140 is configured to interact with the electronic device 120 when the individual generates the neural activity; and wherein the neural monitoring device 100 is further configured to electronically communicate with an electronic network, such as directly and wirelessly to a cloud server or through a wireless connection to the system control unit 140 such that when the control unit 140 is decoupled from the individual, the individual remains able to communicate with the electronic network using the neural activity. The wireless connections can include WIFI connections, Bluetooth, RFID, etc. In another variation, the use of the system control unit 140 is optional, where the internal control unit 130 is configured to wirelessly engage the electronic device 120 and or cloud server. In another variation, the neural monitoring device 100, 130 is configured to electronically communicate with the electronic network independently of the control unit 140.

All existing subject matter mentioned herein (e.g., publications, patents, patent applications) is incorporated by reference herein in its entirety except insofar as the subject matter may conflict with that of the present invention (in which case what is present herein shall prevail). The referenced items are provided solely for their disclosure prior to the filing date of the present application. Nothing herein is to be construed as an admission that the present invention is not entitled to antedate such material by virtue of prior invention.

Reference to a singular item, includes the possibility that there are plural of the same items present. More specifically, as used herein and in the appended claims, the singular forms “a,” “an,” “said” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements or use of a “negative” limitation. Unless defined otherwise, all technical and scientific terms used herein have the same meaning as commonly understood by one of ordinary skill in the art to which this invention belongs.

In understanding the scope of the present disclosure, the term “comprising” and its derivatives, as used herein, are intended to be open-ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. Also, the terms “part,” “section,” “portion,” “member” “element,” or “component” when used in the singular can have the dual meaning of a single part or a plurality of parts. As used herein, the following directional terms “forward, rearward, above, downward, vertical, horizontal, below, transverse, laterally, and vertically” as well as any other similar directional terms refer to those positions of a device or piece of equipment or those directions of the device or piece of equipment being translated or moved. Finally, terms of degree such as “substantially”, “about” and “approximately” as used herein mean a reasonable amount of deviation (e.g., a deviation of up to ±0.1%, ±1%, ±5%, or ±10%, as such variations are appropriate) from the specified value such that the end result is not significantly or materially changed.

This disclosure is not intended to be limited to the scope of the particular forms set forth, but is intended to cover alternatives, modifications, and equivalents of the variations or embodiments described herein. Further, the scope of the disclosure fully encompasses other variations or embodiments that may become obvious to those skilled in the art in view of this disclosure.

1. A method of increasing an autonomy of a individual to operate an external electronic device, the method comprising:
providing a brain-computer interface configured to monitor a neural activity of the individual where the brain-computer interface comprises a neural monitoring device operatively connected to a control unit, where the control unit is configured to produce an output signal for interacting with the external electronic device;
where the brain-computer interface is configured to enter an idle mode wherein the brain-computer interface draws less power than in an active mode; and
receiving an activation signal from the individual to switch the brain-computer interface to an active mode from the idle mode.

2. The method of claim 1, where receiving the activation signal from the individual to switch the brain-computer interface to the active mode from the idle mode occurs without assistance from a caregiver

3. The method of claim 1, further comprising coupling the control unit and the external electronic device to reduce a calibration time for the brain-computer interface when entering the active mode.

4. The method of claim 3, where coupling the control unit and the external electronic device occurs wirelessly.

5. The method of claim 1, wherein the brain-computer interface is configured for coupling to a re-charging supply by the individual.

6. The method of claim 1, wherein the brain-computer interface is configured to transmit operational data from the brain-computer interface to a remote electronic dashboard, where the remote electronic dashboard permits an individual to monitor activity of the brain-computer interface.

7. The method of claim 6, wherein the brain-computer interface is configured to transmit operational data from the brain-computer interface to the remote electronic dashboard wirelessly.

8. The method of claim 1, wherein the brain-computer interface is configured to have a latency of five seconds or less.

9. The method of claim 1, wherein the brain-computer interface is configured to permit the individual to initiate calibration of the brain-computer interface when entering the active mode.

10. The method of claim 1, wherein the individual is a paralyzed individual.
